(19) United States
(12) Patent Application Publication
(10) Pub. No.: US 2025/0375613 A1
(43) Pub. Date: Dec. 11, 2025

(54) CASE FOR USE IN IMPLANTABLE COCHLEAR STIMULATION SYSTEMS AND IMPLANTABLE COCHLEAR STIMULATION SYSTEM EXTERNAL COMPONENT INCLUDING THE SAME

(71) Applicant: ADVANCED BIONICS, LLC, Valencia, CA (US)

(72) Inventor: Rachel E. Potvin, Bellefontaine, OH (US)

(21) Appl. No.: 18/738,593

(22) Filed: Jun. 10, 2024

(51) Int. Cl. A61N 1/36; A61N 1/375; A61N 1/378; H04R 25/00

(52) U.S. Cl. CPC A61N 1/36038; A61N 1/3758; A61N 1/3787; H04R 25/60; H04R 2225/021

(57) ABSTRACT

A case for use with a behind-the-ear sound processor unit includes a main portion including a main portion housing, a cover movable relative to main portion such that the case has an open state and a closed state where the main portion and the cover together define a storage area, a power supply within the main portion housing, an inductive coupler within the main portion housing, and a dock within the storage area, operably connected to the power supply and to the inductive coupler, and configured to dock the behind-the-ear sound processor unit.

### BACKGROUND

### 1. Field

The present disclosure relates generally to implantable cochlear stimulation (“ICS”) systems.

### 2. Description of the Related Art

ICS systems are used to help the profoundly deaf perceive a sensation of sound by directly exciting the intact auditory nerve with controlled impulses of electrical current. Ambient sound pressure waves are picked up by an externally worn microphone and converted to electrical signals. The electrical signals, in turn, are processed by sound processor circuitry, converted to a pulse sequence having varying pulse widths, rates and/or amplitudes, and transmitted to an implanted receiver circuit of the ICS system. The implanted receiver circuit is connected to an implantable electrode array that has been inserted into the cochlea of the inner ear, and electrical stimulation current is applied to varying electrode combinations to create a perception of sound. The electrode array may, alternatively, be directly inserted into the cochlear nerve without residing in the cochlea. A representative ICS system is disclosed in U.S. Pat. No. 5,824,022, which is entitled “Cochlear Stimulation System Employing Behind-The-Ear Sound processor With Remote Control” and incorporated herein by reference in its entirety. Examples of commercially available ICS sound processors include, but are not limited to, the Naida™ CI Q Series and the Naida™ CI M Series behind-the-ear (BTE) sound processors, which are available from Advanced Bionics.

As alluded to above, some ICS systems include an implantable cochlear stimulator (or “cochlear implant”) and external components such as a sound processor (e.g., a BTE sound processor) with sound processor circuitry, a power supply, a microphone that is part of, or is in communication with, the sound processor, and a headpiece that is in communication with the cochlear implant. In some instances, a BTE sound processor may include a sound processor unit with the sound processor circuitry, a power supply unit that may be connected to and disconnected from the sound processor unit, and an ear hook to maintain the sound processor in the intended behind-the-ear location. The headpiece, which is connected to the sound processor unit with a cable that plugs into a headpiece port on the sound processor unit, communicates with the cochlear implant by way of a transmitter (e.g., an antenna coil) on the headpiece and a receiver (e.g., an antenna coil) on the implant. The headpiece and the cochlear implant may also include respective positioning magnets that are attracted to one another, and that maintain the position of the headpiece transmitter over the implant receiver. In other ICS systems, the external components are combined into a single, all-in-one head-mountable off-the-ear sound processor. The sound processor circuitry, microphone, power supply, antenna coil, and positioning magnet are assembled within a single head-mountable housing that is positioned on the cochlear implant recipient's head over the cochlear implant antenna coil. It should be noted here that, as used herein, a BTE sound processor, with or without a head-mounted headpiece, is not a “head-mountable off-the-ear sound processor.”

The present inventor has determined that conventional ICS systems are susceptible to improvement. For example, the present inventor has determined that although some cochlear implant recipients who own a BTE sound processor also desire to have a head-mountable off-the-ear sound processor, obtaining a second sound processor is cost prohibitive.

### SUMMARY

A case for use with a behind-the-ear sound processor unit may include a main portion including a main portion housing, a cover movable relative to main portion such that the case has an open state and a closed state where the main portion and the cover together define a storage area, a power supply within the main portion housing, an inductive coupler within the main portion housing, and a dock within the storage area, operably connected to the power supply and to the inductive coupler, and configured to dock the behind-the-ear sound processor unit.

A head-mountable off-the-ear implantable cochlear stimulation system external component may include a behind-the-ear sound processor (SP) unit and a case. The behind-the-ear SP unit may include a SP unit housing, sound processor circuitry within the SP unit housing, a SP unit headpiece port, and a SP unit power connector configured to connect to a power supply connector on a removable SP power supply. The case may include a main portion including a main portion housing, a cover movable relative to main portion such that the case has an open state and a closed state where the main portion and the cover together define a storage area, a power supply within the main portion housing, an inductive coupler within the main portion housing, and a dock within the storage area, operably connected to the rechargeable power supply and to the inductive coupler, and configured to mate with the SP power connector.

A method of converting a behind-the-ear sound processor (SP) into a head mounted off-the-ear sound processor, the behind-the-ear SP including a SP unit and a SP power supply detachably connected to the SP unit, the SP unit including a SP unit housing, sound processor circuitry within the SP unit housing, a SP unit headpiece port, and a SP unit power connector, may include the steps of detaching the SP power supply from the SP unit and docking the SP unit, with the SP power supply detached therefrom, within a case that includes a power supply and an inductive coupler.

There are a number of advantages associated with such apparatus and methods. By way of example, but not limitation, the case allows a single sound processor unit to for parts of both a BTE sound processor and a head-mountable off-the-ear sound processor. The above described and many other features of the present inventions will become apparent as the inventions become better understood by reference to the following detailed description when considered in conjunction with the accompanying drawings.

### BRIEF DESCRIPTION OF THE DRAWINGS

Detailed descriptions of the exemplary embodiments will be made with reference to the accompanying drawings.

FIG. 1 is a side view of a head-mountable off-the-ear ICS external component in accordance with one embodiment of a present invention.

FIG. 2 is a side view of an exemplary BTE sound processor including the sound processor unit that is part of the ICS external component illustrated in FIG. 1.

FIG. 3 is a side exploded view of the exemplary BTE sound processor illustrated in FIG. 2

FIG. 4 is a side view of a portion of the exemplary BTE sound processor illustrated in FIG. 2.

FIG. 5 is a perspective view of a portion of the exemplary BTE sound processor illustrated in FIG. 2.

FIG. 6 is a perspective view of a portion of the exemplary BTE sound processor illustrated in FIG. 2.

FIG. 7 is a functional block diagram of the exemplary BTE sound processor illustrated in FIG. 2.

FIG. 8 is a side view of a case in accordance with one embodiment of a present invention.

FIG. 9 is a front exploded view of the case illustrated in FIG. 8.

FIG. 10 is a partial section view taken along line 10-10 in FIG. 8.

FIG. 11 is a side view of a portion of the case illustrated in FIG. 8.

FIG. 12 is a side view of a portion of the case illustrated in FIG. 8.

FIG. 13 is a side view of a portion of the case illustrated in FIG. 8.

FIG. 14 is a functional block diagram of the case illustrated in FIG. 8.

FIG. 15 is a front view of a portion of the case illustrated in FIG. 8.

FIG. 16 is a side view showing a portion of a method in accordance with one embodiment of a present invention.

FIG. 17 is a side view showing a portion of a method in accordance with one embodiment of a present invention.

FIG. 18 is a front view showing a portion of a method in accordance with one embodiment of a present invention.

FIG. 19 is a plan view of a cochlear implant.

FIG. 20 is a side view showing the head-mountable off-the-ear ICS external component illustrated in FIG. 1 mounted off-the-ear on a recipient's head over an implanted cochlear stimulator.

FIG. 21 is an exploded side view of a case in accordance with one embodiment of a present invention.

FIG. 22 is a side view of a case in accordance with one embodiment of a present invention in an open state.

FIG. 23 is a front view of the case illustrated in FIG. 22 in a closed state.

FIG. 24 is a front view of a case in accordance with one embodiment of a present invention in an open state.

FIG. 25 is a functional block diagram of the exemplary case illustrated in FIG. 24.

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions.

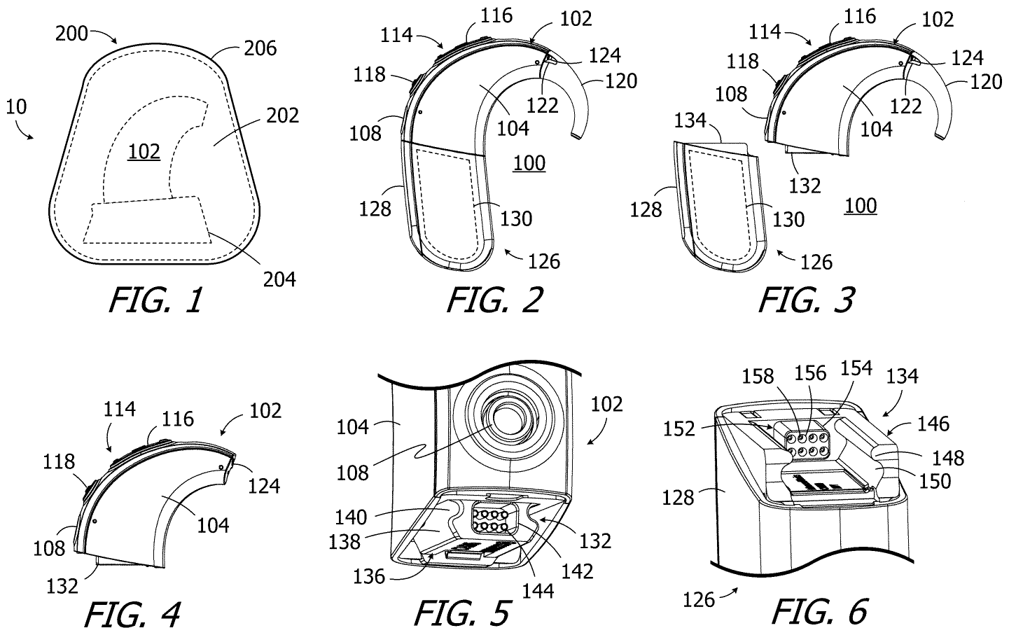

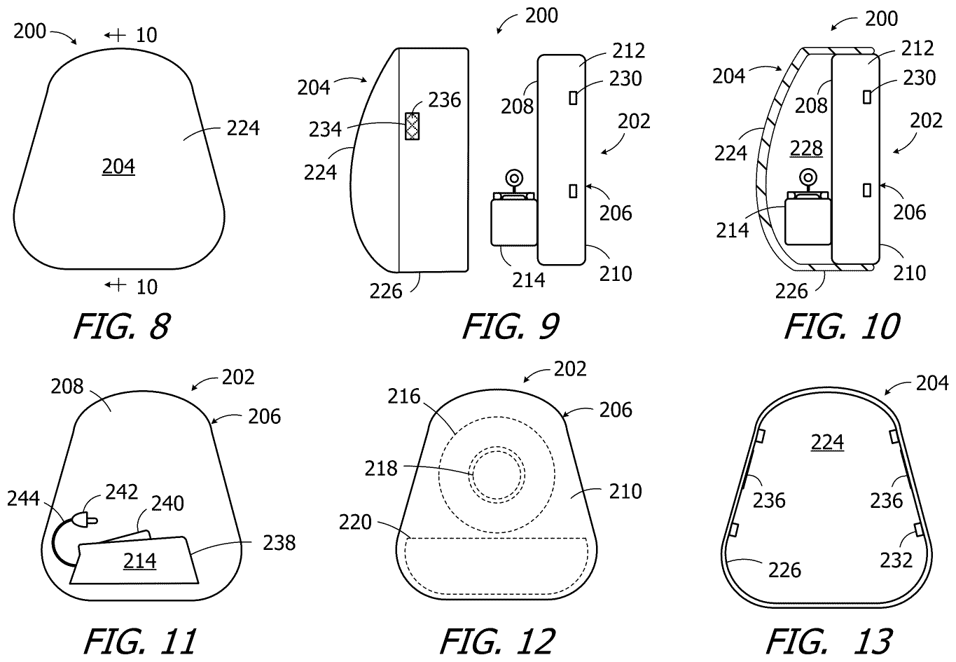

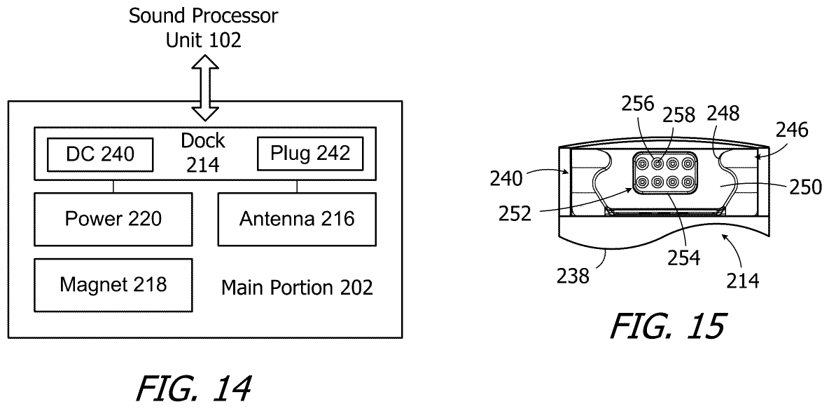

As illustrated for example in FIG. 1, an exemplary ICS external component 10 includes a sound processor unit, such as the sound processor unit 102 that has been separated from the other components of the exemplary BTE sound processor 100 described below with reference to FIGS. 2-7, located within a case, such as the case 200 described below with reference to FIGS. 8-15. Put another way, the sound processor unit 102 may be connected to the other components of the exemplary BTE sound processor 100, and to a headpiece, and used in conventional fashion or, that same sound processor unit 102 may be combined with the case 200 to become part of the ICS head-mountable off-the-ear external component (or “head-mountable off-the-ear sound processor”) 10.

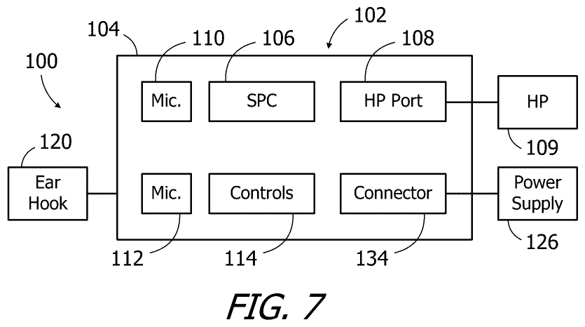

Referring to FIGS. 2-7, an although the present inventions are not limited to use with any particular BTE sound processor, the exemplary BTE sound processor 100 includes a sound processor unit 102 with a processor housing 104 in which and/or on which various components are supported. Such components may include, but are not limited to, sound processor circuitry 106 (FIG. 7), a headpiece port 108 for a headpiece 109 (FIG. 7), front and rear microphones 110 and 112, and a control panel 114. The exemplary control panel 114 has a rocker-type volume switch 116, a program selector switch 118, and an indicator light (not shown). A detachable/removable ear hook 120 with an indentation 122 may be secured to the housing 102. A sound port (not shown) for the rear microphone 112 extends through the housing 104 adjacent to the volume switch 116, and a sound port 124 for the front microphone 110 extends through the housing adjacent to the ear hook indentation 122. The BTE sound processor 100 also includes a detachable/removable power supply unit 126 that supplies power to the sound processor circuitry 106 and other power consuming components of the sound processor unit 102. The power supply 126 includes a power supply housing 128 and one or more batteries or other power supplies 130 (e.g., rechargeable and disposable batteries or other electrochemical cells) that may be removable or non-removable.

The sound processor unit 102 may be electrically and mechanically connected to the power supply 126 by way of the exemplary connectors 132 and 134. Referring more specifically to FIGS. 5 and 6, the exemplary sound processor unit connector 132 includes a connector body 136 with a pair of side projections 138, a pair of side recesses 140 and a receptacle 142. A plurality of tubular electrically conductive contacts 144 are located within the receptacle 142. The exemplary power supply connector 134 includes a connector body 146 with a pair of side projections 148, a pair of side recesses 150 and a plug 152. The plug 152 has a plug body 154 with a plurality of lumens 156 and an electrically conductive contact pin 158 located within each lumen. For example, two of the contact pins 158 may be used for power (+ and −), one of the contact pins may be used for grounding, and the remaining contact pins may be used for fitting and programming.

The exemplary connectors 132 and 134 are respectively configured such that, during connection thereof, the side projections 138 slide into the side recesses 150, the side projections 148 slide into the side recesses 140, and the plug body 152 passes into the receptacle 142. When plug body 152 enters the receptacle 142, the tubular contacts 144 pass into the lumens 156, and the contact pins 158 pass into the tubular contacts 144 and contact the inner surfaces thereof. In some instances, a latch may also be provided to maintain the connectors 132 and 134 in the connected state.

Turning to FIGS. 8-14, the exemplary case 200 includes a main portion 202 and a cover 204. The main portion 202 includes a housing 206 that has a wall 208, which faces the cover 204, a wall 210 that abuts the cochlear implant recipients head during use, and a wall 212. A sound processor unit dock 214 is associated with the wall 208. The exemplary main portion 202 also includes a transmitter (e.g., an antenna coil) 216 that communicates with a cochlear implant receiver (e.g., an antenna coil), a positioning magnet 218 that maintains the position of the headpiece transmitter over the associated implant receiver, and a power supply 220. Particulars such as strength and magnetization of the positioning magnet 218 may vary from recipient to recipient and may be determined by the associated cochlear implant magnet, the recipient's skin flap thickness and physical activity level, and the combined weight of the sound processor unit 102 and case 200. For example, relatively thick recipient skin flaps and physically active lifestyles may require a strong magnet. Some positioning magnets 218 will be diametrically magnetized, while others will be axially magnetized, based on the magnetization of the cochlear implant magnet.

The exemplary cover 204 includes walls 224 and 226 and, when the cover 204 is attached to the main portion 202, a storage volume 228 for the sound processor unit 102 is defined by the cover and the main portion. The cover 204 is shown in an open state in FIG. 9 and a closed state in FIG. 10. The cover 204 may be maintained in the closed state with any suitable instrumentality. By way of example, but not limitation, the case 200 employs a latch arrangement that includes indentations 230 on the main portion wall 212 and protrusions 232 on the cover wall 226. So configured, the cover 204 may be completely separated from the main portion. The main portion may be connected to the cover with a hinge in other implementations, such as those described below with reference to FIGS. 22-25. In either case, the cover may be provided with one or more sound apertures 234 for the microphones 110 and 112 on the sound processor unit 102. The apertures 234 may in some instances be covered with sound permeable moisture resistant material 236. The main portion and/or cover may in some instances include seals (not shown) that result in the case being either waterproof or having an ingress protection (“IP”) rating of at least IP68.

The exemplary dock 214 includes a base 238 that is mounted to the housing wall 208, a dock connector 240 on the base 238, and a headpiece connector 242 that is configured to be received by the headpiece port 108 of the sound processor unit 102. The dock connector 240 is discussed in greater detail below with reference to FIG. 15. The BTE headpiece port 108 may be connected to the transmitter antenna coil 216 by way of the headpiece connector 242 and a cable 244. So connected, the associated sound processor unit (e.g., sound processor unit 102) can facilitate data and power transfer to the associated cochlear implant by way of the antenna 216 in the same way that a sound processor unit facilitates data and power transfer to a cochlear implant by way of a headpiece that is connected to the sound processor unit.

The exemplary power supply 220 may be rechargeable battery or other electrochemical cell, or a replaceable battery, and may be used to power the BTE sound processor unit 102 when it is located within the case 200. The electrical connection is made by way of the dock connector 236. The power supply 220 may also be used to supply power to the associated cochlear implant by way of the transmitter antenna coil 216. Disposable batteries may be employed in some implementations. The power supply 220 may be recharged through inductive coupling to an external charger (not shown) by way of the antenna coil 216 in the illustrated implementation. Batteries may also be replaced in other implementations and, as is discussed below with reference to FIGS. 22 and 23, a plug-in charging port may also be provided in some instances.

Turning to FIG. 15, the exemplary dock connector 240 is substantially similar to the power supply connector 134 of the BTE sound processor 100, thereby allowing the BTE sound processor unit 102 to be connected to the case 200 in the same way that the BTE sound processor unit is connected to the power supply 126. To that end, the exemplary dock connector 240 has a connector body 246 with a pair of side projections 248, a pair of side recesses 250 and a plug 252. The plug 252 has a plug body 254 with a plurality of lumens 256 and an electrically conductive contact pin 258 within each lumen.

The exemplary connectors 132 and 240 are respectively configured such that, during connection thereof, the side projections 138 slide into the side recesses 250, and the side projections 248 slide into the side recesses 140. When the plug body 254 enters the receptacle 142, the tubular contacts 144 pass into the lumens 156, and the contact pins 158 pass into the tubular contacts 144 and contact the inner surfaces thereof.

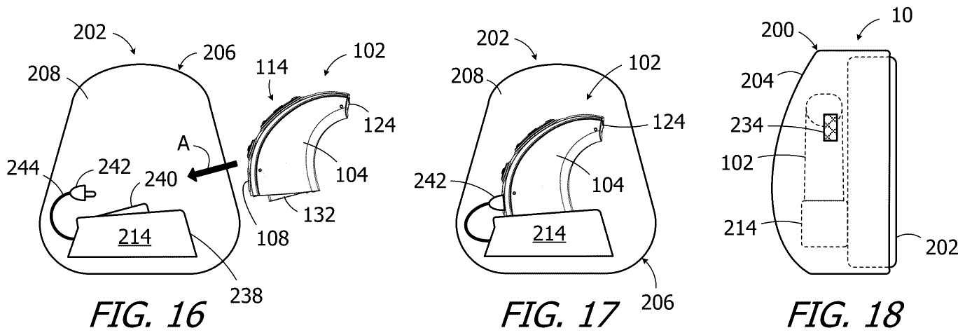

As alluded to above, cases in accordance with the present inventions allow a cochlear implant recipient to form an all-in-one head-mounted ICS external component from the sound processor unit of a BTE sound processor that is configured to be separated from, and re-attached to, the other components of the BTE sound processor. This allows the same single sound processor unit to form part of two different ICS external components, i.e., a BTE sound processor and an all-in-one head-mounted sound processor. One exemplary method of forming a head mounted ICS external component is illustrated in FIGS. 16-18. First, the ear hook 120 and power supply 126 may be removed from the BTE sound processor 100, and the headpiece 109 be disconnected from the headpiece port 108, so that only the BTE sound processor unit 102 remains. The BTE sound processor unit 102 may be positioned relative to the sound processor unit dock 214 in such a manner that the connector 132 is aligned with the connector 240. So aligned, when the BTE sound processor unit 102 is then moved in the direction of arrow A, the side projections 138 of the sound processor connector 132 (FIG. 5) will slide into the side recesses 250 of the dock connector 240 (FIG. 15), and the side projections 248 of the dock connector will slide into the side recesses 140 of the sound processor connector. Continued movement results in the dock connector plug 252 (FIG. 15) being fully inserted into the sound processor receptacle 142 (FIG. 5), with the tubular contacts 144 within the lumens 256 and over the contact pins 258, thereby mechanically and electrically connecting the BTE sound processor unit 102 to the case main portion 202 (FIG. 17). The headpiece connector 242 may then be connected to the headpiece port 108 on the sound processor unit 102. Finally, as shown in FIG. 18, the cover 204 may be positioned over the BTE sound processor unit 102 and secured to the case main portion 202 to complete the exemplary all-in-one head-mountable off-the-ear ICS external component 10.

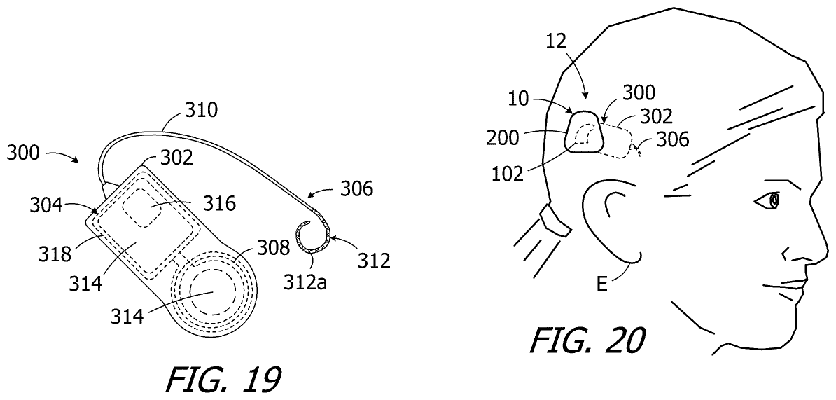

One example of a cochlear implant (or “implantable cochlear stimulator”) with which the external components described herein may be used is generally represented by reference numeral 300 in FIG. 19. The cochlear implant 300 includes a flexible housing 302 formed from a silicone elastomer or other suitable material, a processor assembly 304, a cochlear lead 306, and an antenna coil 308 that may be used to receive data and power by way of an external transmitter (e.g., an antenna coil) that is associated with, for example, the antenna coil 216 of the exemplary case 200. The cochlear lead 306 may include a flexible body 310, an electrode array 312 at one end of the flexible body, and a plurality of wires (not shown) that extend through the flexible body from the electrodes 312a (e.g., platinum electrodes) in the array 312 to the other end of the flexible body. A magnet 314 is located within a region encircled by the antenna coil 308 (e.g., within an internal pocket defined by the housing 302) and ensures that the external antenna coil will be properly positioned relative to the antenna coil 308. The exemplary processor assembly 304, which is connected to the electrode array 312 and antenna 308, includes a printed circuit board 314 with a stimulation processor 316 that is located within a hermetically sealed case 318. The stimulation processor 316 converts the stimulation data into stimulation signals that stimulate the electrodes 312a of the electrode array 312.

Turning to FIG. 20, the exemplary all-in-one head-mountable ICS external component 10, which includes the BTE sound processor unit 102 and the case 200, may be positioned on the recipient's head off-the-ear and over the implanted cochlear implant 300 to form an ICS system 12. The case antenna coil 216 is aligned with the implant antenna coil 308 and the case magnet 218 is aligned with the implant magnet 314. So positioned, the ICS external component 10 is not located behind the ear E of the implant recipient. During use, one or both of the microphones 110 and 112 pick up sound from the environment and convert the sound into electrical impulses. The sound processor circuitry 106 of the sound processor unit 102 filters and manipulates the electrical impulses and sends the processed electrical signals to the antenna coil 216 by way of the dock 214. The signals received by the implant antenna coil 308 are processed by the stimulation processor 316 and passed them through the lead body 310 to the electrode array 312. The electrode array may be wound through the cochlea and provides direct electrical stimulation to the auditory nerves inside the cochlea. This provides the user with sensory input that is a representation of external sound waves which were sensed by the microphones 110 and 112. Power is also transferred to the cochlear implant 300 by way of the antenna coil 216.

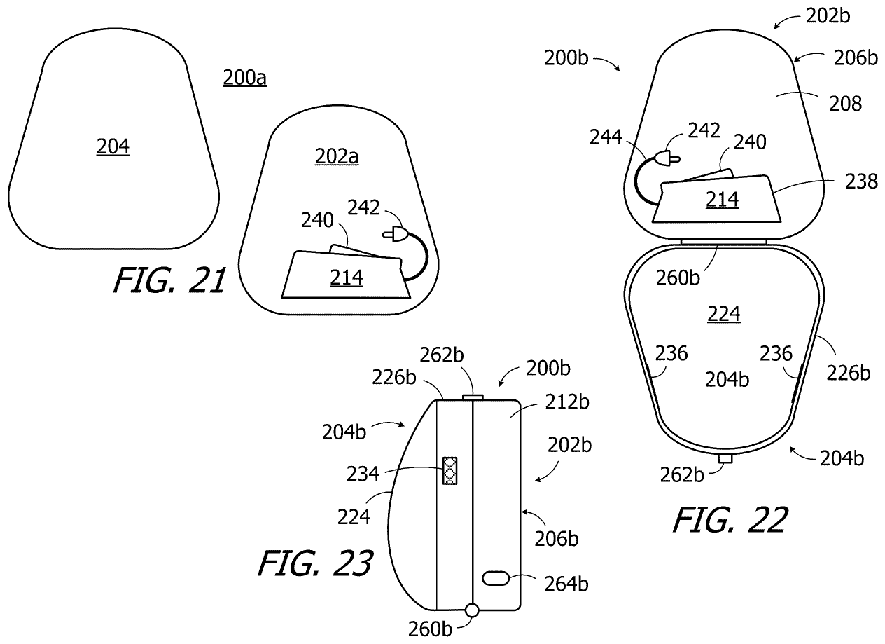

The exemplary case 200 is configured in such a manner that, when worn on the recipient's right side, the microphone 110 faces forwardly. Other embodiments, such as the exemplary case 200a illustrated in FIG. 21 is configured in such a manner that, when worn on the recipient's left side, the microphone 110 faces forwardly. Here, the exemplary case 200a is identical to exemplary case 200 but for the orientation of the dock 214 on the base 202a, which is 180 degrees offset from the orientation of the dock 214 on the base 202.

Another exemplary case is generally represented by reference numeral 200b in FIGS. 22 and 23. The case 200b is substantially similar to case 200 and similar elements are represented by similar reference numerals. Here, however, the main portion 202b is connected to the cover 204b by a hinge 260b and, when the cover 204b is in the closed state (FIG. 23), the cover wall 226b rests on the wall 208 of the main portion housing 206b. The cover 204b is maintained in the closed position by a latch 262b that cooperates with an indentation (not shown) on the main portion wall 212b. Additionally, the case 200b includes a charging port (e.g., a USB charging port) 264b and associated circuitry that may be used to recharge the power supply 220 (FIG. 11). The power supply 220 may also be recharged through electromagnetic induction by way of the antenna coil 216 in the manner discussed above.

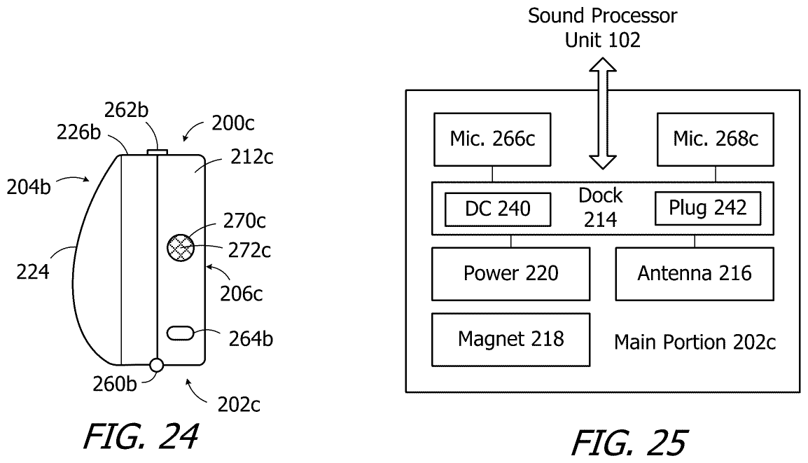

Cases in accordance with the present inventions may also include microphones. To that end, the exemplary case 200c illustrated in FIGS. 24 and 25 is substantially similar to case 200b and similar elements are represented by similar reference numerals. Here, however, the main portion 202c includes forward and rearward facing microphones 266c and 268c within the main portion 202c. The wall 212c of the housing 206c includes front and rear sound ports 270c (only one shown) that are covered with sound permeable moisture resistant material 272c. The microphones 266c and 268c may be connected to the sound processor unit 102 by way of the headpiece port 108 and the headpiece connector 242 on the dock 214. The sound processor unit 102 may also be configured to treat the microphones 266c and 268c in a manner similar to headpiece microphones and to switch between microphones 110 and 112 and the microphones 266c and 268c.

Although the inventions disclosed herein have been described in terms of the preferred embodiments above, numerous modifications and/or additions to the above-described preferred embodiments would be readily apparent to one skilled in the art. By way of example, but not limitation, the cases described herein may also include instrumentalities that are used to retain the case on the recipient's head. Such instrumentalities include, but are not limited to, retention loops for cables and hair clips and removable adhesive pads that may be used by recipients with no hair or very short hair. Headbands may also be used to retain the case on the recipient's in, for example, those instances where the recipient has a relatively thick skin flap and physically active lifestyle. The inventions also include any combination of the elements from the various species and embodiments disclosed in the specification that are not already described. It is intended that the scope of the present inventions extend to all such modifications and/or additions and that the scope of the present inventions is limited solely by the claims set forth below.

1. A case for use with a behind-the-ear sound processor unit, the case comprising:
a main portion including a main portion housing;
a cover movable relative to main portion such that the case has an open state and a closed state where the main portion and the cover together define a storage area;
a power supply within the main portion housing;
an inductive coupler within the main portion housing; and
a dock within the storage area, operably connected to the power supply and to the inductive coupler, and configured to dock the behind-the-ear sound processor unit.

2. The case claimed in claim 1, wherein
the cover is separable from the main portion.

3. The case claimed in claim 1, wherein
the cover and the main portion are connected to one another with a hinge.

4. The case claimed in claim 1, wherein
the inductive coupler comprises an antenna coil.

5. The case claimed in claim 1, wherein
the power supply comprises a rechargeable power supply.

6. The case claimed in claim 5, wherein
the main portion includes a charging port operably connected to the rechargeable power supply.

7. The case claimed in claim 1, wherein
the main portion includes a microphone.

8. The case claimed in claim 1, wherein
the behind-the-ear sound processor unit includes a headpiece port; and
the dock includes a headpiece connector configured to be received by the headpiece port.

9. The case claimed in claim 1, wherein
the dock includes a dock connector with a plug having a plurality of electrically conductive contacts.

10. The case claimed in claim 1, further comprising:
a magnet within the main portion.

11. A head-mountable off-the-ear implantable cochlear stimulation system external component, comprising:
a behind-the-ear sound processor (SP) unit including
a SP unit housing,
sound processor circuitry within the SP unit housing,
a SP unit headpiece port, and
a SP unit power connector configured to connect to a power supply connector on a removable SP power supply; and
a case including
a main portion including a main portion housing,
a cover movable relative to main portion such that the case has an open state and a closed state where the main portion and the cover together define a storage area,
a power supply within the main portion housing,
an inductive coupler within the main portion housing, and
a dock within the storage area, operably connected to the rechargeable power supply and to the inductive coupler, and configured to mate with the SP power connector.

12. The head-mountable off-the-ear ICS system external component claimed in claim 11, wherein
the cover is separable from the main portion.

13. The head-mountable off-the-ear ICS system external component claimed in claim 11, wherein
the cover and the main portion are connected to one another with a hinge.

14. The head-mountable off-the-ear ICS system external component claimed in claim 11, wherein
the inductive coupler comprises an antenna coil.

15. The head-mountable off-the-ear ICS system external component claimed in claim 11, wherein
the power supply comprises a rechargeable power supply.

16. The head-mountable off-the-ear ICS system external component claimed in claim 11, wherein
the main portion includes a charging port operably connected to the rechargeable power supply.

17. The head-mountable off-the-ear ICS system external component claimed in claim 11, wherein
the main portion includes a microphone.

18. The head-mountable off-the-ear ICS system external component claimed in claim 11, wherein
the dock includes a headpiece connector configured to be received by the headpiece port.

19. The head-mountable off-the-ear ICS system external component claimed in claim 11, wherein
the SP power connector includes a receptacle with a plurality of electrically conductive contacts.
the case dock includes a connector with a plug having a plurality of electrically conductive contacts.

20. The head-mountable off-the-ear ICS system external component claimed in claim 11, wherein
the case further comprises a magnet within the main portion.

21-27. (canceled)
